(19) United States
(12) Patent Application Publication
(10) Pub. No.: US 2010/0022648 A1
(43) Pub. Date: Jan. 28, 2010

(54) ORAL USE OF AT LEAST ONE MONOUNSATURATED FATTY ACID FOR HAIR IMPROVEMENT

(75) Inventors: Audrey Gueniche, Rueil Malmaison (FR); Isabelle Castiel, Nice (FR)

Correspondence Address: OLIFF & BERRIDGE, PLC, P.O. BOX 320850, ALEXANDRIA, VA, 22320-4850, US

(73) Assignees: L'OREAL, Paris (FR); NESTEC SA, Vevey (CH)

(21) Appl. No.: 12/518,959

(22) PCT Filed: Dec. 14, 2007
(86) PCT No.: PCT/FR07/52516

§ 371 Date: Sep. 24, 2009

Related U.S. Application Data
(60) Provisional application No. 60/900,990, filed on Feb. 13, 2007.

(30) Foreign Application Priority Data
Dec. 14, 2006 (EP) 06291928.7

(51) Int. Cl. A61K 8/36; A61Q 5/00

(52) U.S. Cl. 514/560

(57) ABSTRACT

The present invention relates to the cosmetic use by the oral route of at least one monounsaturated fatty acid, a salt thereof and/or an ester thereof, preferably petroselinic acid, for improving the quality of the hair and/or the appearance of the hair, in particular for preventing and/or treating the changes of the scalp and/or of the hair that occur with age.

The present invention relates to the use by oral administration of at least one monounsaturated fatty acid in a composition containing a cosmetically or pharmaceutically acceptable medium, as protective agent (treatment or prevention) of the scalp and of the hair.

The hair follicle is formed of perfectly individualized compartments, some of dermal origin (follicular sheath and dermal papilla), others of epithelial nature (outer root sheath, inner sheath, hair shaft and sebaceous gland). The follicular sheath, synthesized by fibroblasts, is primarily an extracellular matrix formed from collagens of type I and III, as well as proteoglycans. Traversed by a network of blood capillaries in the bottom third, it extends to the base of the follicle, via the dermal papilla, a true aggregate of extracellular matrix. The dermal papilla is constituted of particular fibroblasts responsible for local synthesis of prostaglandin E2. A basement membrane, composed of collagen of type IV, laminin of type 1 and 5, fibronectin and heparan sulfate proteoglycans, separates the dermal compartment from the epithelial compartment. The expression profile of laminin 5 is, however, interrupted at the periphery of the bulb, thus emphasizing a regionalization of the dermal-epidermal junction. The epithelial compartment can, in its turn, be divided into four separate regions. At the base of the follicle and surrounding the dermal papilla there is the matrix, a site of intense mitotic activity. This matrix produces, with specific differentiation programs, the three main concentric regions: the outer sheath, the inner sheath and the hair shaft. In the upper portion of the follicle there is the sebaceous gland, formed from particular keratinocytes, the sebocytes, which are responsible for the synthesis of sebum. The upper portion of the outer sheath is also characterized by the presence of Langerhans cells, veritable sentinels of the immune system, and Merkel cells filled with neuropeptides, whose role is still obscure (Review in “La vie révélée du follicule de cheveu humain” (The life revealed of the human hair follicle); Bernard B A, Medecine Sciences Vol 22(2), 2006).

The applicant has over the course of several years conducted numerous clinical tests and has thus been able to determine the symptoms associated with aged hair. These symptoms are in particular a change in appearance of the fiber (hair that is fine, dull, limp, lusterless, graying), hair that tends to fall out, and is renewed less quickly. These manifestations result from disorganization of the collagen network in the dermis of the scalp, from the appearance of glycation products (1) which stiffens the dermis around the hair follicle leading to adverse effects for the resident follicles and the implantation of new hair, and reduction of the synthesis of sebum, thus increasing the dull and lusterless appearance of the hair and promoting hair loss (2, 3).

(1) Monnier V M, Cerami A. ACS Symposium Series 215 (1983): 431-449

(2) Jaworsky C, Kligman A M, Murphy G F. Br J Dermatol 127 (1992): 239-246

(3) Sueki H, Stoudemayer T et al. Acta Dermatol Venereol 79 (1999): 347-350

To have healthy, vigorous and abundant hair throughout one's life is the ambition of most men and women. Numerous products for treatment of the hair are available. However, to date there is no composition for the oral route for combating the changes of the scalp and/or of the hair that occur with age, for protecting the “hair capital”.

The applicant has demonstrated that the monounsaturated fatty acids might be able to combat the changes of aged hair Chronological aging is a natural phenomenon that is manifested progressively and earlier or later depending on the individual. The manifestations of aging of the hair can however be accelerated by endogenous factors, stress, illness etc., or exogenous factors such as pollution, UV exposure etc.

The monounsaturated fatty acids have been described for various applications, such as the hydration of dry skin (EP 0 709 084 of L'Oreal).

EP 0 116 439 (Suntory) discloses that certain fatty acids have strong activity of inhibition of the 5α-reductase of the bacterial flora of the scalp, and so can be used in tonics for the treatment of dandruff and itching of the scalp. The action of these fatty acids is therefore directed at the bacterial flora of the scalp.

U.S. Pat. No. 4,097,604 (Oxford Hill) states that a salt of various fatty acids is active against the pathogenic microorganisms of the oral cavity, thus making it possible to reduce the effect of these bacteria on appearance of periodontoses. The action of these fatty acids is in this case limited to the oral flora.

EP 0 355 842 (Sansho Seiyaku) describes a care cream intended to prevent the pigmentation caused by overproduction of melanin; said cream can contain petroselinic acid.

EP 1 013 178 (Unilever) describes the use of petroselinic acid as antiinflammatory agent and for treating the cutaneous signs of aging (wrinkles, flabby skin, old age spots).

BP 888 773 (NESTLE) describes a composition containing petroselinic acid, used for the treatment or prevention of inflammations and/or modulation of lipids in the superficial tissues. However, the cosmetic use of petroselinic acid for combating the changes of the scalp and/or of the hair that occur with age is neither described, nor suggested there.

Moreover, none of these documents predicted the effects of the monounsaturated fatty acids on the quality of the hair fiber and thus of the hair.

Thus, the present invention relates to the use, notably the cosmetic use, by the oral route, of at least one monounsaturated fatty acid, a salt thereof or an ester thereof, for preventing and/or treating the changes of the scalp and/or of the hair that occur with age.

According to a particular embodiment, the changes of the scalp and/or of the hair that occur with age mean changes connected with the aging process, notably changes that are not mediated by an inflammatory process. In particular, said changes mean changes linked solely to the aging process, whether of the chronological aging or photoaging type.

As said inflammatory process that is not considered in the present invention, we may notably mention that resulting from an infection, an allergy, an autoimmune disease, or exposure to irritants.

Thus, according to a particular embodiment, the present invention relates to the use, notably the cosmetic use, by the oral route, of at least one monounsaturated fatty acid, a salt thereof and/or an ester thereof, notably petroselinic acid, for preventing and/or treating the changes of the scalp and/or of the hair that occur with age and are not mediated by an inflammatory process, in particular not mediated by an inflammatory process induced by exposure to an irritant.

It is understood within the scope of the present invention that “cosmetic use by the oral route” covers the use of products administered by the oral route, said products being, for example, in the form of a food supplement or of functional food, producing an effect on the scalp and/or on the hair in aesthetic or comfort terms, or for beauty purposes, for example for the purposes of protection, maintaining in good condition, changing the appearance, and notably for beautifying.

The changes of the scalp, especially the changes of the scalp that occur with age are notably fibroses of the tissues surrounding the hair follicles.

The applicant has in fact been able to demonstrate that the monounsaturated fatty acids lead to:
a restructuring of the collagen network that can notably be visualized by staining with Masson's Trichrome on a skin section from a subject receiving coriander oil as a supplement; in fact, the action of this oil is to reduce collagenase and elastase activity (measured by enzyme assay);
a decrease in glycated proteins found in the dermis and the epidermis (decrease in carboxymethyl-lysine derivatives);
a decrease in markers of stress and of inflammation (such as Hsp 70 and TNF-alpha, which can be measured with specific antibodies) since the oxidative stress of the tissue surrounding the hair follicles leads to a change of the hair, or even hair loss.

They also prevent changes to the dermis by tissue enzymes such as collagenases, elastases and gelatinases and thus lead to a skin profile, especially of the scalp, that is healthy, without fibrosis, favorable to the growth of hair of good quality.

The use of at least one monounsaturated fatty acid, a salt thereof or an ester thereof also makes it possible to limit hair loss and/or increase hair density.

Hair density is determined as a function of the number of hairs for a given area of the scalp.

According to one embodiment, the use according to the invention is intended for combating the changes of aged hair. It is also intended for protecting aged hair.

In particular, the use according to the invention makes it possible to prevent and/or limit the formation of fine and/or dull and/or brittle and/or limp hair.

The use according to the invention also makes it possible to improve the quality of keratin fibers, notably by promoting the growth of hair that is shiny and/or thick and/or vigorous.

Monounsaturated fatty acid means, in the sense of the present invention, a fatty acid containing a single double bond.

It relates more particularly to long-chain fatty acids. The monounsaturated fatty acids suitable for the invention are the monounsaturated fatty acids having 12 to 22 carbon atoms.

The monounsaturated fatty acids can be in the form of acid, or salt, or in the form of derivatives, notably esters or amides of fatty acids.

When in the form of salts, the monounsaturated fatty acids can more particularly be cosmetically acceptable salts, i.e. inorganic salts such as salts of ammonium or of alkali metals (lithium, potassium, sodium), of alkaline-earth metals (magnesium, calcium), or salts of aluminum. In particular, the monounsaturated fatty acids are in the form of calcium salts.

The fatty acids can also be in the form of esters. They can then be esterified with glycerol (mono-, di- or tri-acyl), an alcohol such as methyl and ethyl alcohols, a sugar, a tocopherol, a tocotrienol, a sterol or a fatty alcohol.

As monounsaturated fatty acids, we may mention petroselinic acid (or C₁₈ delta-6-cis-octadecenoic acid), palmitoleic acid (or C₁₆ cis-9-hexadecenoic acid) or oleic acid (C₁₈ cis-9-octadecenoic acid).

It is understood that the fatty acids are selected taking into account the intended purpose of the composition containing them, and notably its method of administration, oral or via the respiratory passages. By respiratory passages, we mean the upper respiratory passages (such as the nasal fossae, the sinuses, the mouth).

Among the monounsaturated fatty acids, in particular oleic acid and petroselinic acid are used. Petroselinic acid is quite particularly suitable for the invention.

According to a variant of the invention, the monounsaturated fatty acid or acids are used in isolated form, i.e. after extraction from their source.

According to another variant of the invention, the monounsaturated fatty acid or acids are obtained from vegetable extracts such as oils.

Thus, the invention notably relates to the use of an oil rich in monounsaturated fatty acid.

We can notably use oils rich in petroselinic acid, selected more particularly from oils from Umbelliferae.

“Oil rich in petroselinic acid” means an oil containing at least 40% of petroselinic acid.

The Umbelliferae are plants whose flowers are arranged in umbels; the species particularly rich in petroselinic acid are the Umbelliferae Apiaceae and Araliaceae. The plants of the genus Thapsia are also sources of petroselinic acid (Avato et al., Lipids, 2001, 36, 845). The species preferably used in the invention are coriander, chervil, carrot, celery, cumin, caraway, parsley and dill. Umbellifer oil used according to the invention can be extracted from the seed of these Umbelliferac, for example by grinding or pressing, then refining. Umbellifer oil has a content of petroselinic acid that varies depending on the umbellifer seed from which it is extracted. For one and the same umbellifer, the content of petroselinic acid also varies according to the country of origin of the umbellifer and according to the degree of extraction, which can be more or less complete.

Petroselinic acid is also an abundant compound (about 48%) in the oil from seed of Gernium sanguneum (Tsevegsuren et al., Lipids, 2004, 39, 571).

Thus, according to one embodiment, the use according to the present invention is such that petroselinic acid is used in the form of umbellifer oil or oil of Gernium sanguneum.

According to one embodiment, a composition according to the present invention is such that the content of monounsaturated fatty acid, a salt thereof and/or an ester thereof is between 0.0001 and 30 wt. % relative to the total weight of the composition.

The content of monounsaturated fatty acid or its derivatives in the compositions for the oral route for use according to the invention will be such that the daily dose of said monounsaturated fatty acid, a salt thereof and/or an ester thereof is between 0.5 and 2500 mg/day, notably between 10 and 1200 mg/day, particularly between 5 and 500 mg/day.

According to a particular embodiment of the invention, the content of monounsaturated fatty acids in the compositions for use by the oral route is such that the daily dose is for example between 5 and 50 mg/day or between 150 and 1200 mg/day and notably between 150 and 600 mg/day.

The oral compositions for use according to the invention preferably have an edible carrier.

For ingestion, numerous embodiments of oral compositions and notably of food supplements are possible. They are formulated by the usual methods to produce coated pills, hard capsules, soft capsules, gels, emulsions, tablets. In particular, the active substance or substances according to the invention can be incorporated in any other forms of food supplements or enriched foods, for example food bars, or compacted or uncompacted powders. The powders can be diluted in water, in soda, milk products or soya derivatives, or can be incorporated in food bars.

Particularly suitable food or pharmaceutical carriers are milk, yoghurt, cheese, fermented milk, milk-based fermented products, ice creams, products based on fermented cereals, milk-based powders, formulas for children and infants, food products such as confectionery, chocolate, cereals, feeds for animals in particular domestic animals, tablets, capsules or lozenges, oral supplements in dry form.

Of course, the oral compositions according to the invention can additionally contain several other actives.

These actives can be selected from vitamins B1, B3, B5, B6, B8, B12, C, D, E, or PP, niacin, carotenoids, polyphenols and minerals, amino acids, catechins, oligo-proanthocyanidins, sulfated amino acids, omega 3 and 6 polyunsaturated fatty acids and gelatin.

According to a particular embodiment, these actives can be selected from those suitable for combating the aging of the scalp and for improving the appearance of the hair, and in particular taurine, vitamin B6, cysteine, vitamin B1 (biotin), vitamin B12 (bepanthene) and gelatin can be used.

The oral compositions can in addition contain an antioxidant complex comprising vitamins C and E, and at least one carotenoid, notably a carotenoid selected from β-carotene, lycopene, astaxanthin, zeaxanthin and lutein, flavonoids such as the catechins, hesperidin, proanthocyanidins notably in the form of blackcurrant seed oil and anthocyanins.

The oral compositions can additionally contain at least one probiotic, a prebiotic or a mixture of probiotic and a mixture of prebiotics. As probiotic microorganisms, we may notably mention Lactobacillus johnsonii or Lactobacillus paracasei.

According to another of its aspects, the invention relates to a cosmetic method for improving the quality of the hair and of the hair fiber comprising at least one stage of ingestion of an oral composition containing, in a physiologically acceptable carrier, at least one monounsaturated fatty acid and/or an ester thereof and/or a salt thereof.

Preferably, the monounsaturated fatty acid is petroselinic acid.

The cosmetic method of the invention can comprise a single administration. According to another embodiment, administration is repeated for example 2 to 3 times daily for one day or more and generally for a prolonged duration of at least 4 weeks, or even 4 to 15 weeks, with one or more breaks if necessary.

According to yet another of its aspects, the invention relates to the use of at least one monounsaturated fatty acid, a salt thereof or an ester thereof for the manufacture of a cosmetic or dermatological composition intended for treating and/or preventing pathologies of the scalp and/or of the hair, in particular of the scalp and/or of aged hair.

According to yet another of its aspects, the invention relates to a monounsaturated fatty acid, a salt thereof or an ester thereof for the treatment and/or the prevention of pathologies of the scalp and/or of the hair, in particular of the scalp and/or of aged hair.

### EXAMPLE 1

### Monodose Gel

| | wt. % |
|---|---|
| Active principle | |
| Blackcurrant seed oil | 10 |
| Coriander oil | 10 |
| Lycopene | 10 |
| Lactobacillus johnsonii | 10¹⁰ CFU |
| Excipients | |
| Sugar syrup | 50 |
| Maltodextrin | 17 |
| Xanthan gum | 0.8 |
| Sodium benzoate | 0.2 |
| Water | Q.S. 100 |

A dose of 200 to 400 ml per day can be taken.

### EXAMPLE 2

### Capsule

| | mg/capsule |
|---|---|
| Oil of fennel | 300 |
| Grapeseed oil | 200 |
| Lactobacillus paracasei | 10¹⁰ CFU |
| Glycerol | 150 |
| Magnesium stearate | 0.02 |
| Natural flavor | 0.01 |

One to three of these capsules can be taken per day.

### EXAMPLE 3

A vitamin complex containing 60 mg of vitamin C, 100 μg of vitamin E and 6 mg of β-carotene is added to the formulation in Example 2.

### EXAMPLE 4

A vitamin complex containing 100 mg of vitamin C, 100 μg of vitamin E and 6 mg of lycopene per capsule is added to the formulation in Example 2.

### EXAMPLE 5

A vitamin complex containing 100 mg of vitamin B1, B6, B12, 100 μg of taurine and 6 mg of gelatin per capsule is added to the formulation in Example 2.

### EXAMPLE 6

### Capsule

| | mg/capsule |
|---|---|
| Vitamin C | 60 |
| Carrot oil | 300 |
| Coriander oil | 200 |
| Glycerol | 150 |
| Magnesium stearate | 0.02 |
| Natural flavor | q.s. |

One to three of these capsules can be taken per day.

### EXAMPLE 7

A vitamin complex containing 60 mg of vitamin C, 100 μg of vitamin E and 6 mg of β-carotene is added to the formulation in Example 6.

### EXAMPLE 8

A vitamin complex containing 100 mg of vitamin C, 100 μg of vitamin E and 6 mg of lycopene per capsule is added to the formulation in Example 6.

### EXAMPLE 9

A vitamin complex containing 100 mg of vitamin B1, B6, B12, 100 μg of taurine and 6 mg of gelatin per capsule is added to the formulation in Example 6.

### EXAMPLE 10

### Investigation In Vitro of the Effect of Petroselinic Acid on the Formation of Glycation Products in Dermal Cells

In the test described below, the applicant demonstrated that petroselinic acid makes it possible to significantly reduce the formation of glycation products in cultures of dermal fibroblasts.

Dermal fibroblasts are cultured by the usual techniques that are well known by a person skilled in the art.

The glycation products are quantified by densitometric analysis of the spots in immunodetection of the glycation products (Dot Blot method), relative to a control (culture of fibroblasts without addition of the product).

In one case the culture of fibroblasts is supplemented with aminoguanidine, as reference product, based on its action of significantly reducing the amount of glycation product in the experimental conditions of this test. As shown in Table 1 below, we detect the formation of 42% of glycation products relative to the control. Thus, aminoguanidine reduces the amount of glycation product relative to the control by 58%. This result can therefore validate the experimental conditions of this test.

In another case, the culture of fibroblasts is supplemented with petroselinic acid. As shown in Table 1 below, we detect the formation of 73% of glycation products relative to the control, thus indicating a significant decrease (27%) in the formation of glycation products relative to the control.

TABLE 1

| Treatment | % GP relative to the control |
|---|---|
| Control | 100 |
| Aminoguanidine 1 mg/ml | 42* |
| Petroselinic acid 0.885 mM | 73* |

*significant difference relative to the control p < 0.01
GP = glycation products

Thus, this test demonstrates that petroselinic acid allows a significant reduction in the formation of glycation products in the dermis, thus combating the changes of the dermis due to age.

The monounsaturated fatty acids according to the invention can therefore be used for preventing and/or treating the age-related changes of the dermis, and in particular the changes of the scalp and/or of the hair that occur with age.

1. A cosmetic method by the oral route for preventing and/or treating the changes of the scalp and/or of the hair that occur with age comprising the oral administration of at least one monounsaturated fatty acid, a salt thereof or an ester thereof.

2. The method as claimed in claim 1, wherein fibroses of the tissues surrounding the hair follicles are prevented and/or treated.

3. The method as claimed in claim 1, wherein hair loss is limited and/or hair density is increased.

4. The method as claimed in claim 1, wherein aged hair is protected.

5. The method as claimed in claim 4, wherein the formation of fine and/or dull and/or brittle and/or limp hair is prevented and/or limited.

6. The method as claimed in claim 1, wherein the quality of keratin fibers is improved.

7. The method as claimed in claim 6, wherein the growth of hair that is shiny and/or thick and/or vigorous is promoted.

8. The method as claimed in claim 1, wherein the monounsaturated fatty acid is used in isolated form or in a vegetable extract.

9. The method as claimed in claim 1, wherein the content of monounsaturated fatty acid, a salt thereof and/or an ester thereof is between 0.0001 and 30 wt. % relative to the total weight of the composition.

10. The method as claimed in claim 1, wherein the content of monounsaturated fatty acid, a salt thereof and/or an ester thereof in the composition for the oral route is such that the daily dose of said monounsaturated fatty acid, a salt thereof and/or an ester thereof is between 0.5 and 2500 mg/day.

11. The method as claimed in claim 1, wherein the monounsaturated fatty acid is petroselinic acid.

12. The method as claimed in claim 11, wherein petroselinic acid is used in the form of umbellifer oil or oil of Gernium sanguneum.

13. The method as claimed in claim 12, wherein the umbellifer oil is selected from the oils of seeds of coriander, chervil, carrot, celery, cumin, caraway, parsley and dill.

14. The method as claimed in claim 1, wherein said monounsaturated acid is used in combination with at least one active selected from vitamins B1, B3, B5, B6, B8, B12, C, D, E, or PP, niacin, carotenoids, polyphenols and minerals, amino acids, catechins, oligo-proanthocyanidins, sulfated amino acids, omega 3 and 6 polyunsaturated fatty acids and gelatin.

15. A cosmetic method for improving the quality of the hair and of the hair fiber comprising the ingestion of an oral composition containing at least one monounsaturatecd fatty acid and/or a salt thereof and/or an ester thereof.

16. The method as claimed in claim 15, wherein the monounsaturated fatty acid is petroselinic acid.
